(19) United States
(12) Patent Application Publication
(10) Pub. No.: US 2006/0079822 A1
(43) Pub. Date: Apr. 13, 2006

(54) Ankle brace

(76) Inventor: Knud Hjorth, Gadstrup (DK)

Correspondence Address: DAVID A. GUERRA, 317 - 649 MARSH ROAD N.E, CALGARY, AB, T2E 5B4, CA

(21) Appl. No.: 10/543,261

(22) PCT Filed: Jan. 12, 2004
(86) PCT No.: PCT/DK04/00010

§ 371 Date: Jul. 25, 2005

(30) Foreign Application Priority Data
Mar. 6, 2003 (DK) PA 2003 00346

(51) Int. Cl. A61F 5/00

(52) U.S. Cl. 602/28; 602/5; 602/27

(57) ABSTRACT

Ankle brace with a sole as a basis. On the sole is fastened a vertical heel counter by means of a click arrangement and the heel counter ends with a joint, which is freely moveable in all directions. In continuation of the moveable joint is on the joint fastened a vertical bar, mounted on the top with an in high adjustable fitting, on which is mounted a semicircular double counter, which from behind rounds equal forward on both sides of the calf of the leg. The inner counter rests direct on the calf of the leg and is connected to the outer counter in both sides by a moveable joint, by which it can move in upward and downward direction in relation to the outer counter. Next to these joints are in both sides mounted a hook, from where in both sides an elastic adjustable strap crosses over the instep to the sole and is fastened under this.

The invention relates to an ankle brace—Orthosis—which can be used in all cases, where nerves, muscles and similar in the legs or elsewhere in the nervous system are damaged in such a way, that the person will not be able to raise the front part of the foot sufficient to have a normal walk, the so called drop feet

The ankle brace is constructed so, that the sole to be put into the shoe, forms the base of the ankle brace.

On the sole is mounted a heel counter by means of a click arrangement. On the heel counter is mounted a joint, which is constructed thus, that the sole and the heel counter freely are able to move in all directions compared with the upper part of the ankle brace and this means, that the foot has free movement in the ankle joint in all directions and so the person will achieve a free and natural walk and the crossed straps cause, that the foot is placed in a normal position after every step in contrast to all other known ankle braces, where the movement of the foot is fixed to a movement back and forth, but not sideways. The movement of the ankle joint in all directions causes furthermore, that the steel bar and the double counter are able to rock completely to the rear by means of which, there is no form of hindrance, when the foot is put into the shoe.

The assembling of the heel counter and the sole by means of a click arrangement causes, that the different sizes of sole and counter as a matter of course can be clicked together and be adjusted to the individual shoe and this in connection with, that the upper double counter is adjustable and that the length of the straps by moving the hole in the strap, which is hitched on the double counter, can be adjusted individually, causes, that the ankle brace immediately can be adjusted to the shoe of the individual person.

With the double counter an improvement has been achieved, that where other counters glide up and down direct upon the calf of the leg, the movement between the inner and the outer counter will cause, that the movement up and down against the calf will be caught by the movement of the joint.

We can refer to the following well-known ankle braces:

DE number 297 04 669 U 1

DE number 199 41 368 A 1

both having an ankle brace with a bar on each side of the foot and fastened thus, that the sideway movements are inflexible, and

U.S. Patent No. 5,860,423

in which the ankle brace on the heel counter is fastened in 2 points by which the sideway movements are not possible.

As regards DE 199 41 368 A 1 it is stated, that the upper counter can be taken to pieces by which it will be possible to force the ankle braces from each other in order to make it easier to put them on.

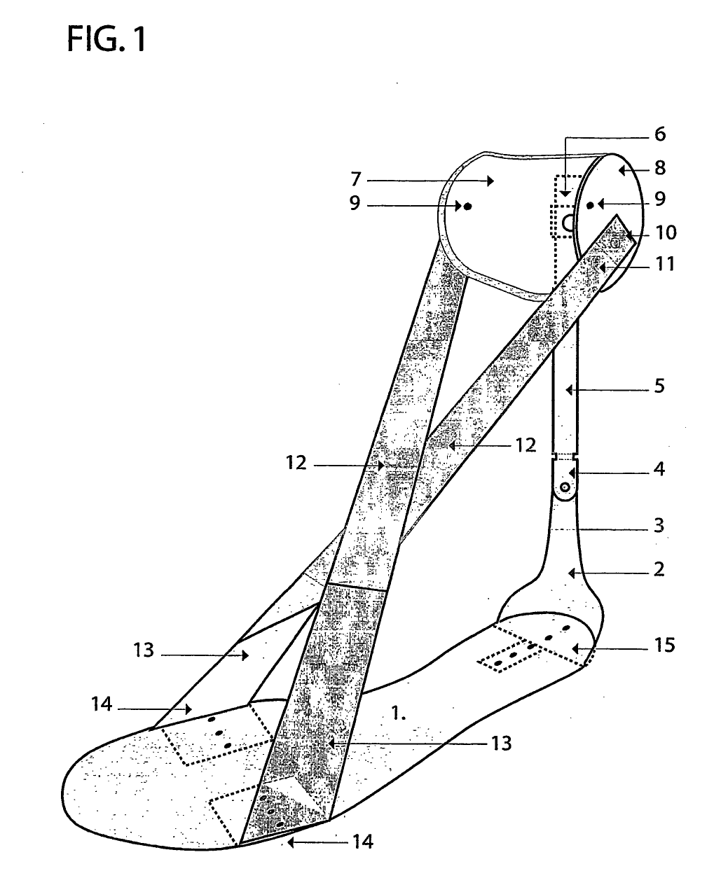

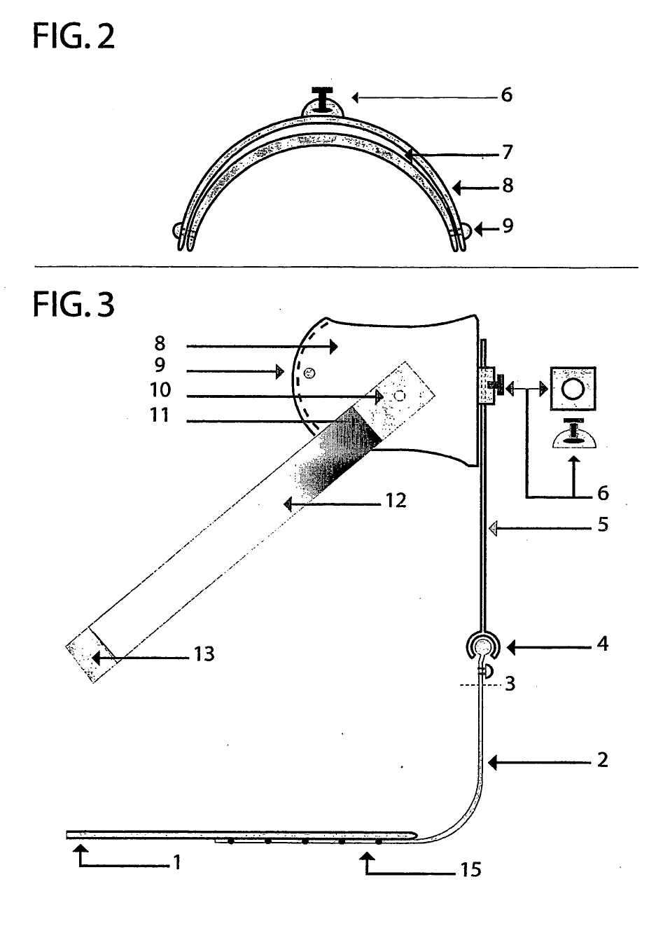

The invention will be explained more precisely in the following, referring to the drawings FIG. 1 showing the ankle brace from in front, FIG. 2 showing the double counter and fittings and FIG. 3 showing the ankle brace from the side.

The ankle brace consists of a sole (FIG. 1: 1, FIG. 3: 1), which will be fitted to the customers footwear. The sole will be provided with a vertical heel counter (GIG. 1: 2, FIG. 3: 2), which will be fitted to the heel counter of the shoe. This heel counter ends about 1 centimetre over the heel counter of the shoe, marked by a line (FIG. 1: 3, FIG. 3:.3).

The joining of the sole and the vertical heel counter is made by a click system, that means that on the underside of the sole, small downward turned knobs are cast. In the heel counter going under the sole (FIG. 1: 15, FIG. 3: 15) are made holes, which diameter and intervals are as the knobs thus, that the sole and the heel counter can be clicked together. The heel counter ends with a joint (FIG. 1: 4, FIG. 3: 4), which allows movements backwards and forwards and at the same time to both sides, which means full movement In vertical continuation of this joint is mounted a steel bar (FIG. 1: 5, FIG. 3: 5) On the steel bar is mounted a sliding fitting (FIG. 1: 6, FIG. 2: 6, FIG. 3: 6), which can be moved up and down the steel bar and can be secured in the high wanted by means of a stretching screw (FIG. 2: 6, FIG. 3: 6).

On this gliding fitting is mounted a semicircular forward turned counter (FIG. 1: 8, FIG. 2: 8, FIG. 3: 8) thus, that the centre of the circle is mounted on the sliding fitting and so, that the ends of the counter go equally forward on both sides of the leg. This counter will because of the direct connection with the sole in the shoe (FIG. 1: 2, 4, 5, 6, 15), (FIG. 2: 6) and (FIG. 3: 2, 4, 5, 6, 15) follow the movements of the shoe heel while walking.

In order to counteract these movements, the outer counter (FIG. 1: 8, FIG. 2: 8, FIG. 3: 8) is provided with an inner counter (FIG. 1: 7, FIG. 2: 7), which lies upon the calf of the leg. The inner counter is fastened to the outer counter in both sides with a moveable joint (FIG. 1: 9, FIG. 2: 9, FIG. 3: 9) by which the up and down going movements are counteracted.

On the outer side of the outer counter are in both sides mounted a hook arrangement (FIG. 1: 10, FIG. 3: 10) or a piece of Welco-belt for fastening in both sides a 2-3 centimetre wide elastic strap (FIG. 1: 11-14), which is composed of the pieces FIG. 1: 11-13, FIG. 3: 11-13, of which the piece FIG. 1: 11, FIG. 3: 11 is an inelastic strap. After that follows the piece FIG. 1: 12, FIG. 3: 12 which is an elastic strap and there after follows the piece FIG. 1: 13, FIG. 3: 13 which is an inelastic strap. The latter is mounted under the sole at FIG. 1: 14 by use of a click arrangement like that, used under the sole and the heel counter (FIG. 1:15, FIG. 3: 15).

In the last-mentioned case knobs will be cast under the sole in both sides, so that the holes in the bottom of the strap (FIG. 1: 14) fit the knobs in both sides under the sole.

By this one achieves to be able to use the single strap on both sides of the foot, just by an inversion of the strap.

The piece of the strap FIG. 1: 13, FIG. 3: 13 is placed inside the shoe, while the piece of the strap FIG. 1:12, FIG. 3: 12 comes out of the shoe and lies freely and turns half round over the instep and goes up to the piece of the strap FIG. 1: 11, FIG. 3: 11, which is fastened at FIG. 1: 10, FIG. 3: 10.

1. Ankle brace to put on a foot to set right, that the foot cannot by itself be raised sufficient by walking, the so called drop feet
The ankle brace consists of a lower part, namely the sole (FIG. 1: 1) with one with a click system (FIG. 1: 15, FIG. 3: 15) fastened heel counter (FIG. 1: 2) and also an upper part, namely the bar (FIG. 1: 5, FIG. 3: 5) with a fastened adjustable fitting (FIG. 2: 6, FIG. 3: 6) with one to the fitting fastened adjustable double counter (FIG. 1: 7, 8, FIG. 2: 7, 8). Upper and lover part are in front put together by 2 elastic adjustable straps, which crosswise over the instep go from the hook devise (FIG. 1: 10, FIG. 3: 10 on the double counter to the sole (FIG. 1: 14), where the straps are fastened under the sole with a click system and upper and lower part of the brace are on the backside of the leg (the calf) put together in a moveable joint (FIG. 1: 4)
characterised in, that the joint (FIG. 1: 4) is constructed thus, that it is freely moveable in all directions.

2. Ankle brace according to claim 1
characterised in, that the sole (FIG. 1: 1) and the heel counter (FIG. 1: 2) are put together by means of a click system (FIG. 1:15, FIG. 3: 15)

3. Ankle brace according to claim 1 or 2
characterised in, that the fixed 2 counters of the double counter (FIG. 1: 7, 8, FIG. 2: 7, 8) freely are able to move up and down between each other by 2 joints (FIG. 1: 9, FIG. 2: 9)
